(19) United States
(12) Plant Patent Application Publication
(10) Pub. No.: US 2006/0041979 P1
(43) Pub. Date: Feb. 23, 2006

(54) Guinea impatiens plant named 'Ovation Suncatcher Salmon'

(75) Inventor: Lyndon W. Drewlow, Lompoc, CA (US)

Correspondence Address: THE WEBB LAW FIRM, P.C., 700 KOPPERS BUILDING, 436 SEVENTH AVENUE, PITTSBURGH, PA, 15219, US

(73) Assignee: Oglevee, Ltd., Connellsville, PA (US)

(21) Appl. No.: 11/210,277

(22) Filed: Aug. 23, 2005

Related U.S. Application Data
(60) Provisional application No. 60/603,581, filed on Aug. 23, 2004.

(51) Int. Cl. A01H 5/00

(52) U.S. Cl. PLT/318

(57) ABSTRACT

A new and distinct cultivar of New Guinea Impatiens plant named ‘Ovation Suncatcher Salmon’, characterized by its mounded to semi-upright plant habit; freely branching growth habit; freely flowering habit with rounded flowers positioned above the foliage; large salmon-colored flowers; variegated leaves; and tolerance to full sun conditions and low and high temperatures.

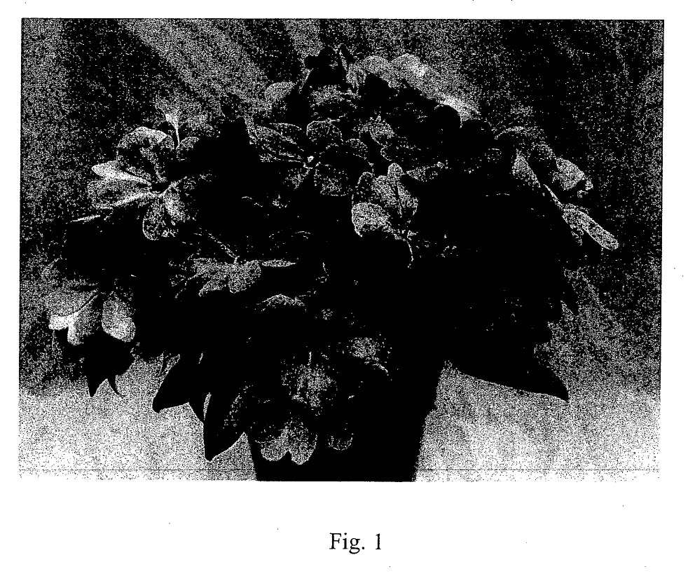

### CROSS-REFERENCE TO RELATED APPLICATIONS

This application claims the benefit of U.S. Provisional Plant Patent Application No. 60/603,581 filed Aug. 23, 2004, which is hereby incorporated by reference in its entirety.

### BOTANICAL CLASSIFICATION

Impatiens hawkeri

### VARIETAL DENOMINATION

‘Ovation Suncatcher Salmon’

### BACKGROUND OF THE INVENTION

The present invention comprises a new and distinct cultivar of Impatiens hawkeri, known by the cultivar name ‘Ovation Suncatcher Salmon’. The new variety is a product of a planned breeding program conducted in Ashtabula, Ohio. The purpose of the breeding program was to create early flowering cultivars with a large flower size, numerous salmon flowers, variegated leaves and flowers over the leaf canopy. The new variety originated from a cross-pollination of a proprietary selection of Impatiens hawkeri identified as code number 97-735-3, not patented, as the female seed parent with a proprietary selection of Impatiens hawkeri identified as code number 97-730-4, not patented, as the male pollen parent. The cultivar ‘Ovation Suncatcher Salmon’ was discovered and selected as a flowering plant within the progeny of the stated cross in a controlled environment. Asexual reproduction of the new cultivar by terminal cuttings has shown that the unique features of the new variety are stable and reproduced true to type in successive propagations.

The following traits have been repeatedly observed and are determined to be the unique characteristics of ‘Ovation Suncatcher Salmon’. Characteristics that distinguish ‘Ovation Suncatcher Salmon’ as a new and distinct cultivar from others known to the breeder include:

1) Very rounded flowers due to the overlap of the petals;

2) Flowers of the color of 47C;

3) Large flowers, 80 to 85 mm in diameter;

4) Variegated leaves with a color of 147A with areas of a color of 19C around the midrib;

5) Free-branching habit;

6) Mounded to semi-upright growth habit;

7) Early to flower, finishing in 8 to 10 weeks in a 15 cm pot;

8) Ability to tolerate both high temperatures and full sun; and

9) Ability to tolerate cool night temperatures of 5 to 10° C.

Plants of the new Impatiens can be compared to plants of the Impatiens cultivar ‘Ovation Suncatcher Orange’, disclosed in U.S. Plant Pat. No. 14,486. In side-by-side comparisons conducted in Lompoc, Calif., plants of the new variety differed from plants of the cultivar ‘Ovation Suncatcher Orange’ in the following characteristics:

1) Flowers of the new variety were larger than the flowers of ‘Ovation Suncatcher Orange’;

2) Flowers of the new variety had a color of 47C, whereas flowers of ‘Ovation Suncatcher Orange’ had a color of 43B;

3) Leaves of the new variety had a color of 147A with areas of a color of 19C around the midrib, whereas leaves of ‘Ovation Suncatcher Orange’ had a color of 147A with areas of a color of 153C around the midrib;

4) Plants of the new variety had a mounded to semi-upright habit, whereas plants of ‘Ovation Suncatcher Orange’ had a mounded habit; and

5) Peduncles of the new variety were shorter than the peduncles of the cultivar ‘Ovation Suncatcher Orange’.

### BRIEF DESCRIPTION OF THE DRAWING

The accompanying colored photographic drawing illustrates the new variety with the colors being as nearly true as is possible to obtain in colored illustrations of this type.

### DESCRIPTION OF THE PLANT

The cultivar ‘Ovation Suncatcher Salmon’ has not been observed under all possible environmental conditions. The phenotype may vary somewhat with variations in environment such as temperature, day length and light intensity. However, there is no variance in genotype.

In the following description, color references are made to The Royal Horticultural Society Colour Chart, 1995 Edition, except where general terms of ordinary dictionary significance are used. Plants were grown under commercial practice during the late winter through early spring of 2003 in Lompoc, Calif., in a corrugated polycarbonate-covered greenhouse with 30% shade covering. During the production of the plants, day temperatures ranged from 21 to 24° C.; night temperatures ranged from 16 to 18° C.; and light levels ranged from 4000 to 5000 foot candles of light. Plants used in the following description were grown in 15 cm containers for about 8 to 10 weeks after planting rooted cuttings.
Propagation:
   Type cutting.—Terminal cuttings.
   Time to initiate roots.—Summer: About 10 days at 21° C. Winter: About 12 days at 21° C.
   Time to produce a rooted cutting.—Summer: About 21 days at 21° C. Winter: About 21 days at 21° C.
   Root description.—Fibrous, white in color.
   Root habit.—Freely branching, dense.
Plant description:
   General appearance.—Mounded to semi-upright plant habit; numerous rounded flowers positioned above the foliage.
   Plant height at flowering.—20 to 25 cm.
   Plant width (spread).—30 to 35 cm.
   Growth and branching habit.—Freely branching habit; dense and full plant habit; typically 10 or more lateral branches develop per plant. Pinching (removal of terminal apex) is usually not required. Vigorous growth habit.
   Lateral branch description.—Length: About 15 to 20 cm. Diameter: About 8 to 10 mm. Internode length: About 5 to 6 cm. Color: 146B with a tint of 184C.
   Foliage description.—Arrangement: Alternate or whorled; simple. Length: About 9 to 10 cm. Width: About 3 to 3.5 cm. Shape: Lanceolate. Apex: Acuminate. Base: Acute. Margin: Finely serrated with fine cilate. Texture, upper and lower surfaces: Glabrous. Venation pattern: Pinnate. Color: Developing leaves, upper surface: 146A. Developing leaves, lower surface: 146B. Fully expanded leaves, upper surface: 147A with an area of 19C around the midrib. Fully expanded leaves, lower surface: 148B with an area of 11C around the midrib. Venation, upper surface: 146A. Venation, lower surface: 146A. Petiole length: About 10 to 12 mm. Petiole diameter: About 3 mm. Petiole color: 148B with a tint of 64D.
Flower description:
   Flower type and habit.—Single, large, rounded, flat and salmon-colored flowers. Freely flowering, usually about one flower per leaf axil. Flowers are positioned above the foliage and typically face upward or outward. Flowers last about two weeks on the plant depending on environmental conditions. Petals not persistent. Flowers not fragrant.
   Flowering season.—Year-round under greenhouse conditions. In the garden, flowering from spring until fall. Flowering indeterminate and continuous.
   Flower diameter.—About 8 to 8.5 cm.
   Flower buds.—Length: About 2 cm. Width: About 1.5 cm. Shape: Ellipsoidal Color (just before opening): 50A.
   Petals.—Quantity: Single, five. Length: About 3.5 to 4.5 cm. Width: About 4.0 to 5.0 cm. Shape: Cordate. Apex: Rounded with moderate to deep indentations. Base: Acute. Margin: Entire. Texture, upper and lower surfaces: Glabrous, satiny. Color: When opening, upper surface: 50A. When opening, lower surface: 54B. Fully opened, upper surface: 47C fading to 52C. Fully opened, lower surface: 51C.
   Spur.—Length: About 5.5 cm. Color: 185D.
   Peduncles.—Length: About 4.0 cm. Angle: About 45° from vertical. Strength: Strong. Texture: Smooth, glabrous. Color: 148D with a tint of 184C.
   Reproductive organs.—Androecium: Stamen number: Five fused at anthers, filaments free, hooded. Anther length: About 5 mm. Anther shape: Obovate. Anther color: 54B. Pollen amount: Abundant. Pollen color: White. Gynoecium: Pistil number: One, five-segmented. Pistil length: About 5 mm. Stigma color: 155C. Style color: 155C. Ovary color: 146A.
Fruit/Seed development: Fruit and seed development has not been observed.
Disease/Pest resistance: Plants of the new variety have not been observed to date to be resistant to pathogens and pests common to Impatiens.
Temperature tolerance: In outdoor nursery trials in Connellsville, Pa., plants of the new variety have been observed to be tolerant to full sun conditions and high temperatures (32 to 38° C.) during the summer. In outdoor nursery trials in Lompoc, Calif., plants of the new Impatiens have been observed to be tolerant to low night temperatures from about 5 to 10° C.

I claim:

1. A new and distinct variety of Impatiens named “Ovation Suncatcher Salmon’ as herein described and illustrated.
